(19) United States
(12) Plant Patent Application Publication
(10) Pub. No.: US 2012/0137397 P1
(43) Pub. Date: May 31, 2012

(54) Odontocidium burgundian 'Too Sweet'

(50) ;

(76) Inventor: James McCully, Hilo, HI (US)

(21) Appl. No.: 12/928,004

(22) Filed: Nov. 30, 2010

(51) Int. Cl. A01H 5/00

(52) U.S. Cl. PLT/311

(57) ABSTRACT

A new variety of orchid plant of the Oncidiinae Intergenerics, Odontocidium, named Odontocidium Burgundian ‘Too Sweet’, distinguished particularly by a clean pleasing arrangement of burgundy and white flowers with a pleasant fragrance on a raceme that is desirable in size for the size of the plant and growing quickly to sexual maturity.

### CROSS-REFERENCE TO RELATED APPLICATIONS

Not Applicable

### STATEMENT REGARDING FEDERALLY SPONSORED RESEARCH OR DEVELOPMENT

Not Applicable

### LATIN NAME OF THE GENUS AND GREX OF THE PLANT CLAIMED

The Latin name of the genus and grex of the plant claimed is Odontocidium Burgundian.

### VARIETY DENOMINATION

The present invention comprises a new and distinct cultivar of Odontocidium orchid, and hereinafter referred to by the cultivar name Odontocidium Burgundian ‘Too Sweet’.

### BACKGROUND OF THE INVENTION

Odontocidium is the nothogenus comprising intergeneric hybrids of the two orchid genera Odontoglossum and Oncidium in the subtribe Oncidiinae. Odontocidium consists of about 58 species and Oncidium consists of about 330 species. The first properly identified and registered hybrid of Odontocidium was created and registered by Lairesse in 1905. The hybrid was registered as Odontocidium Lairesseae (Oncidium fuscatum×Odontoglossum crispum). Due to the varying percentages of the parent genera and large number of species that can be involved that make up Odontocidium and the gene expression in their progeny, Odontocidium varies in morphology and size. All exhibit sympodial growth habit.

Odontocidium breeding is typically done from sexual methods. Asexual propagation of Odontocidium is often done in aseptic tissue culture from apical or axillary shoots.

The new cultivar was discovered within the progeny of a cross made in Hilo, Hi. on Jan. 23, 2002. The Inventor selected Odontocidium Burgundian ‘Too Sweet’ as a single plant from a population of over 200 plants grown in Hilo, Hi. Odontocidium Burgundian ‘Too Sweet’ was re-flowered for evaluation and submitted by the Inventor to a commercial tissue culture laboratory in Bangkok, Thailand on Dec. 1, 2006 for propagation through aseptic tissue culture technique.

Other seedlings from the same cross that was used to produce Odontocidium Burgundian ‘Too Sweet’ were sold by the Inventor and may have been commercially available from others. The sibling plants were sold without specific epithet to individuals and other nurseries. To the Inventor's knowledge, the only other named clones of the cross are Odontocidium Burgundian ‘Velvet Underground’ and Odontocidium Burgundian ‘Delectable’ and are owned by the inventor. The inventor has reason to believe that Odontocidium Burgundian ‘Too Sweet’ is superior to the other seedlings in growth and foliage, number of flowers, shape, fragrance and color as well as the number of spikes and spiking habit. Plants of the grex Odontocidium Burgundian are sold as plants of the grex without specific epithet.

### BRIEF SUMMARY OF THE INVENTION

The following traits have been repeatedly observed and are determined to be basic characteristics of the new cultivar which in combination distinguish this Odontocidium orchid as a new and distinct cultivar:

Flowers of the new cultivar have petals and sepals that are burgundy turning creamy white basally. The labellum is burgundy fading to a spray paint pink before turning into a creamy white picotee. The anther cap is cream in color, sometimes with speckles of burgundy. The callus is mainly burgundy, turning yellow basally. The column is creamy white at its base and becomes burgundy apically. The underside of the column is cream with yellow and burgundy spots. The column wings are narrowly lamellate and burgundy in color. The flowers are rigid and heavily scented. The natural horizontal spread is 4.5 centimeters (cm); natural vertical spread is 6 cm; dorsal sepal width 1 cm; dorsal sepal length 2.6 cm; petal width 1 cm; petal length 2.3 cm; lateral sepal width 0.9 cm; lateral sepal length 3 cm; labellum width 3.1 cm; labellum length 3.3 cm. The plant usually produces two inflorescences on the first bloom with and average of 8 flowers per inflorescence.

Inflorescence is a basal raceme, upright or arching, and approximately 32 cm tall. The plant is relatively compact and the size of the inflorescence is appropriate to the overall size of the plant and typical pot size for this cultivar.

There are generally six leaves on the mature pseudobulb, two from the apex and two from each side of the base of the pseudobulb. The apical leaves measure 27 to 37.5 cm and are 3.1 to 3.7 cm wide. The leaves from the base of the pseudobulb measure from 26 to 30 cm long and 3.4 to 3.7 cm wide. Axillary leaves, or bracts, are sometimes present below the leaves at the base of the pseudobulb. When present, there is usually one on either side of the pseudobulb and measure 12 to 20 cm long and 2.1 to 2.7 cm wide. The shape of the leaf is lanceolate acuminate and semi-conduplicate at the base. The texture and substance is very slightly rugose due to parallel vein striation, waxy and thin. The plants of Odontocidium Burgundian ‘Too Sweet’ grow to maturity and flower in approximately 18 months.

Plants of the new cultivar have not been observed under all possible environmental conditions. The phenotype may vary significantly with variations in the environment such as temperature, light intensity, and day length, without however any change in genotype.

Plants of the new cultivar differ primarily from the plants of the parent cultivars in flower color, panicle size, rate of growth and speed to maturation. Perhaps the closest commercial comparison to the new cultivar can be made to seedling-derived Odontocidium orchids which are genetically heterogeneous, and typically lack uniformity in growth, vigor, plant habit, and flower quality. Since this reference point has inconsistent characteristics, a direct comparison for Odontocidium Burgundian ‘Too Sweet’ is not available. The new cultivar is a single genotype asexually propagated via tissue culture; thus, its combined horticultural characteristics listed above are uniform and predictable.

### BRIEF DESCRIPTION OF THE DRAWINGS

The drawings show the colors of the new variety as nearly true as possible with an illustration of this type. In the drawings:

FIG. 1 is a view of the entire plant to reveal the growth habit and general characteristics with its flowers, which is typical of this new variety.

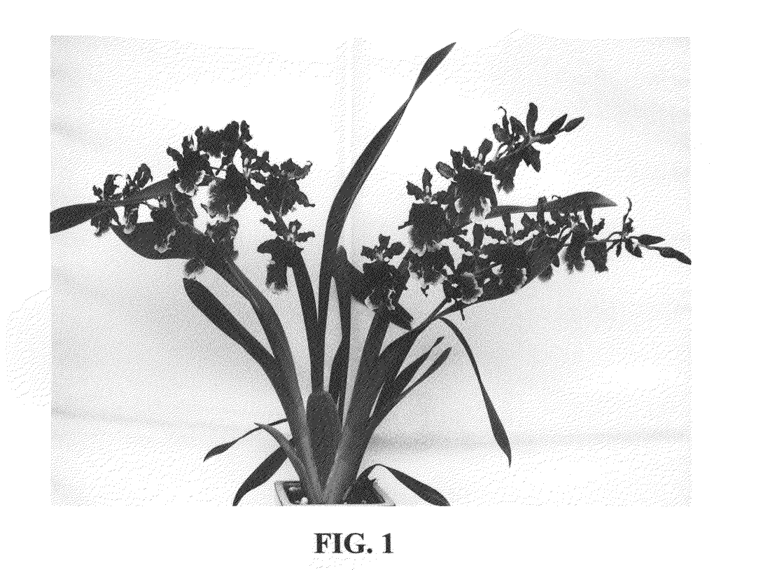

FIG. 2 is a close up view of the flower to show its shape.

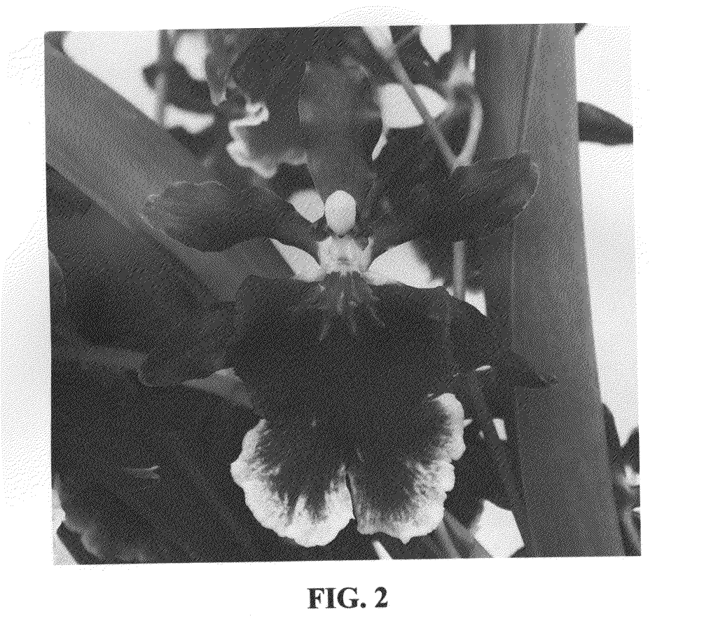

### DETAILED BOTANICAL DESCRIPTION

All color references are measured against the Pantone® Color System. Colors and numerical measurements are approximate as plant growth and development depends on environmental conditions and cultural practices such as light level, temperature, water quality, fertilization formula and rate, among others, without, however, any variance in genotype.

Plants used for this description are 18 months in vivo and grown in square, black plastic pots measuring 3.25″ at the top of the pot and tapering down to 2.5″ at the bottom of the pot with a height of 3.75″, grown in a poly-film covered greenhouse in Hilo, Hi., where day temperatures range from 75 to 85 degrees and night temperatures range from 55 to 65 degrees Fahrenheit. Light levels are approximately 2500 foot candles. This information reflects the annual variations for the area.
Botanical classification: OdontocidiumBurgundian ‘Too Sweet’
Parentage: Seedling selected from the cross of the following:
   Seed parent.—Oncidium schroederianum.
   Pollen parent.—Odontocidium Harry Sutton.
Propagation:
   Type.—Asexual propagation by aseptic tissue culture through axillary shoot initiation.
   Time to initiate and elongate roots in-vivo.—about 45 days.
   Time to produce fully rooted young plant.—about 240 days.
Root description: Velamen covered roots, approximately 2 mm in diameter, whitish tan in color with yellowish green growing tip. The exact shades of white and yellow/green vary greatly with minimal changes in environmental conditions.
Plant description: Under appropriate growing conditions, plants of the new cultivar attain a mature size of about 37.5 cm in height (top of leaf plane) and about 3.7 cm in width. The height from soil level to the top of the inflorescences averages 32 cm.
Foliage description:
   Arrangement.—Sympodial growth habit.
   Quantity of leaves per growth.—Each sympodial growth has shown six leaves, two on the apex of the pseudobulb and four from the axis at the base of the pseudobulb.
   Leaf length.—Apical leaves measure from 27 to 37.5 cm in length. The leaves at the base of the pseudobulb may be as short as 26 cm to and as long as 30 cm.
   Leaf width.—The width of the largest leaf measures up to 3.7 cm.
   Leaf shape.—The shape is lanceolate acuminate, entire, apex acute and slightly recurved with a semi-conduplicate base. The leaf margins are smooth, parallel venation apparent, texture and substance of both the upper and lower surfaces of the leaf is very slightly rugose, waxy and thin.
   Pseudobulb shape.—The pseudobulb shape is a laterally compressed ovoid.
   Pseudobulb length.—The pseudobulb is approximately 5 cm long.
   Pseudobulb width.—The pseudobulb is approximately 4.5 cm wide and 2 cm thick.
   Foliage color.—Under the described growing conditions, the foliage color on both the upper and under sides of the leaves is similar to Pantone® 574C (green) of the Pantone® Color System. There is no vein color distinction. The pseudobulb color is Pantone® 384UP (green).
Flower/inflorescence description:
   Description.—Inflorescence is an upright or arching raceme, terete, approximately 32 cm in length and 0.3 cm in width on a first bloom plant, typically occurring on the second pseudobulb and grown in square black plastic pots measuring 3.25″ on top, 2.5″ on the bottom with a height of 3.75″. The glabrous peduncles are approximately 12 cm in length to the first flower. The pedicels are approximately 1.6 to 2.7 cm in length and 0.2 cm in diameter. Although somewhat pliable when physically bent, they are fairly rigid and strong, supporting each flower sufficiently. The angle of the pedicel is 75-90° giving the flowers proper spacing to display themselves without overcrowding. The texture of the pedicels is fairly smooth and the coloration is light green (Pantone® 375UP) at the base and dark burgundy (Pantone® 490C) at the top half towards the flower. Each raceme averages 8 flowers. Typically, there two inflorescences per pseudobulb, but frequently three inflorescences per pseudobulb have been observed on this cultivar, the third as an apical inflorescence. The flowers begin approximately halfway up the raceme and are spaced around 2 cm apart. The size of the inflorescence is appropriate to the overall size of the plant and typical pot size for this cultivar. The above description is of a first bloom plant in the pot sized as outlined with 2 pseudobulbs. If additional pseudobulbs are grown, each pseudobulb will produce 1-3 racemes with and average of 8 flowers. The flower buds are conical acute, 0.5 to 0.7 cm in diameter and 1.6 to 2.1 cm in length. The surface is glabrous and the coloration is Pantone® 490C (dark burgundy) overlaid in some areas by light (Pantone® 367PC). The flowers are bilaterally symmetrical, which is typical of most orchids. Each flower consists of 3 sepals and 3 petals. The lower petal is modified and referred to as the labellum and/or lip. The upper sides of the sepals and petals are burgundy in color (Pantone® 7428C). The lower sides of the sepals and petals are burgundy (Pantone® 7428C) with irregular streaking of gray (Pantone® 4C) and creamy white (Pantone® 691C) basally for approximately 0.5 cm. The dorsal sepal is lanceolate, apex apiculate and reflexed for 0.2 cm, margins undulate. The lateral sepals are similar in shape though slightly smaller in length and width. The base of the sepals is creamy white (Pantone® 691C) for 0.3 to 0.4 cm in a triangular pattern with the tip pointing to the apex. Petals are lanceolate acuminate, apex reflexed for 0.3 to 0.4 cm with somewhat undulate margins and have a creamy white band for 0.15 cm at the base. Sepal and petal texture is satiny. The labellum (lip) is a compressed ovoid, bilobed with serrate and undulate margins. The lip lamina is somewhat rectangular shaped at its base. The texture of the upper surface of the lip is velvety. The lower surface of the leaf has a satin texture. The throat of the lip is yellow (Pantone® 1225C) for 0.1 cm, bordered on two sides spots of white (Pantone® 691C) that are about 0.4 cm wide by 0.2 cm high. Below this, the lip is deep burgundy red (Pantone® 504C) showing pink (Pantone® 224PC) venation, for approximately 2.7 cm before it fades into fuchsia (Pantone® 222C) for 0.35 cm and finally lined with white (Pantone® 691C) picotee around bottom edge of lip for 0.1cm. The lower surface of the lip is deep burgundy red (Pantone® 504C) basally, fading to fuchsia (Pantone® 222C) and lined with a white (Pantone® 691C) picotee. The callus of the labellum begins as a swelling in the throat of the lip which bifurcates into multiple individual lamella that rise up approximately 0.15 cm and range from 0.1 to 0.4 cm long and equal to or less than 0.1 cm wide. The texture of the apical surfaces of the calli are dentate, the sides are rigid and smooth. The color for all surfaces of the callus is deep burgundy red (Pantone® 504C).
   Dimensions.—Flower dimensions are: natural horizontal spread is 4.5 cm; natural vertical spread is 6 cm; dorsal sepal width is 1.0 cm; dorsal sepal length is 2.6 cm; lateral sepal width is 0.9 cm; lateral sepal length is 3 cm; petal width is 1.0 cm; petal length is 2.3 cm; labellum width is 3.1 cm; labellum length is 3.3 cm.
   Coloration.—The dominant flower color that appears on the upper and lower surfaces of the sepals and petals is burgundy, Pantone® 7428C. The interior white bands on the tepals are Pantone® 691C. The irregular gray streaking on the lower surfaces of the tepals is Pantone® 4C. The dominant color of the lip is deep burgundy red, Pantone® 504C, lined with fuchsia, Pantone® 222C, and white, Pantone® 691C. The upper and side colors of the column at its base is creamy white, Pantone® 7499UP, and burgundy for the apical half, Pantone® 7428C. The lower surface of the column is white, Pantone® 7499UP, with a yellow spot in the center, Pantone® 7405C, and a burgundy spot, Pantone® 7433C) within that yellow spot. The pollinia are completely concealed by the anther cap, and if it is removed the pollinia may be extracted. The pollinia are yellow, Pantone® 7408C, but can range in shades of yellow as the plant ages.
Quantity of flowers and time to flower:
   Flower longevity.—The flowers can last longer than 8 weeks on the plant with good environmental conditions. A first bloom plant will produce around 16 flowers depending on environmental conditions. Since this plant has been grown for pot plant purposes, cut flower longevity has not been observed.
   Natural flowering season.—This plant has appeared to be free flowering although the dominant flowering season is fall (September, October, November, December).
Fragrance: A strong fragrance has been detected by the Inventor.
Reproductive organs: The stamens, style and stigmas are fused into a single short structure called the column, possessing one terminal anther with pollen grains united to pollinia, which are covered by an anther cap. The stigma is located under the column behind the pollinia. The ovary is inferior, with three carpels being present.
   Column.—The column is erect with narrowly lamellate wings on either side of the stigma, 2 mm wide by 6 mm long.
   Pollinia.—Two oval masses of pollen are present, about 0.75 mm in diameter and 1.5 mm long.
   Stigma.—The stigma is concave, round, with a high gloss, and sticky. The length of the stigma is 4 mm and the width is 4 mm. The stigma is a translucent clear color, therefore taking on the creamy white color of the column, Pantone® 7499UP.
   Ovary.—The ovary is about 1.6 to 2.7 cm long by 1.25 mm in diameter and light green in color (Pantone® 375UP) on basal half, burgundy (Pantone® 490C) in color on the apical half.
   Seed.—Seed production has not been observed.
Disease resistance: Resistance or susceptibility to known pathogens of Odontocidium has not been observed on plants grown under commercial production conditions.
General observations: Plants of Odontocidium Burgundian ‘Too Sweet’ produce a pleasing arrangement of burgundy and white flowers with a pleasant fragrance on a raceme that is desirable in size for the size of the plant. The flowers are long lasting and nicely shaped. The plant grows quickly to sexual maturity.

What is claimed is:

1. A new and distinct variety of orchid plant named Odontocidium Burgundian ‘Too Sweet’, substantially as illustrated and described herein.
